(19) United States
(12) Patent Application Publication
(10) Pub. No.: US 2006/0116542 A1
(43) Pub. Date: Jun. 1, 2006

(54) Metathesis catalyst and process

(75) Inventors: David Stephen Brown, Hercules, CA (US); Josiane Marie-Rose Ginestra, Richmond, TX (US)

Correspondence Address: SHELL OIL COMPANY, P O BOX 2463, HOUSTON, TX, 772522463, US

(73) Assignee: Shell Oil Company

(21) Appl. No.: 11/287,604

(22) Filed: Nov. 28, 2005

Related U.S. Application Data
(60) Provisional application No. 60/631,777, filed on Nov. 30, 2004.

(51) Int. Cl. C07C 6/00

(52) U.S. Cl. 585/643

(57) ABSTRACT

An olefin metathesis process and a catalyst composition suitable for such process comprising (a) rhenium, (b) one or more metal(s) from Columns 5 and 6 of the Periodic Table, and (c) a support made from an alumina; wherein surface area of the catalyst is at least 200 m²/g as determined by ASTM D-3663-03.

### REFERENCE TO RELATED APPLICATION

This application claims the benefit of U.S. Provisional application Ser. No. 60/631,777, filed Nov. 30, 2004, the entire disclosure of which is herein incorporated by reference.

### FIELD OF THE INVENTION

This invention relates to supported mixed-metal catalysts useful in olefin metathesis reactions and to a metathesis process employing such catalyst.

### BACKGROUND OF THE INVENTION

Metathesis, also known as disproportionation, is a reaction in which one or more olefinic compounds are catalytically converted into other olefin(s) of a different molecular weight(s) through exchange between olefin molecules of groups situated at the double bond of the olefin molecule. The disproportionation of an olefin with itself to produce an olefin of a high molecular weight and an olefin of a lower molecular weight is referred to as self-disproportionation.

Another type of disproportionation involves the cross-disproportionation of two different olefins to form still other olefins. One example is the reaction of one molecule of 2-butene with one molecule of 3-hexene to produce two molecules of 2-pentene. Another example is 1-butene disproportionated to ethylene and 3-hexene. 3-Hexene may further undergo a double bond isomerization to form 2-hexene as a side product.

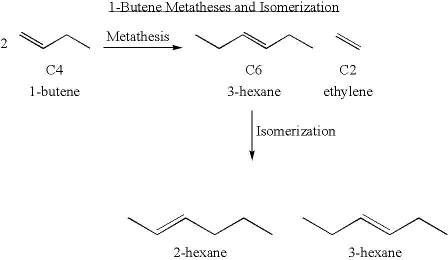

Another example is 1-hexene disproportionated to ethylene and 5-decene. In a side reaction 1-hexene may isomerize to form 2-hexene which may self-metathesize to form side products of 2-butene and 4-octene or cross-metathesize to form propylene, 2-pentene, 2-heptene, and 4-nonene.

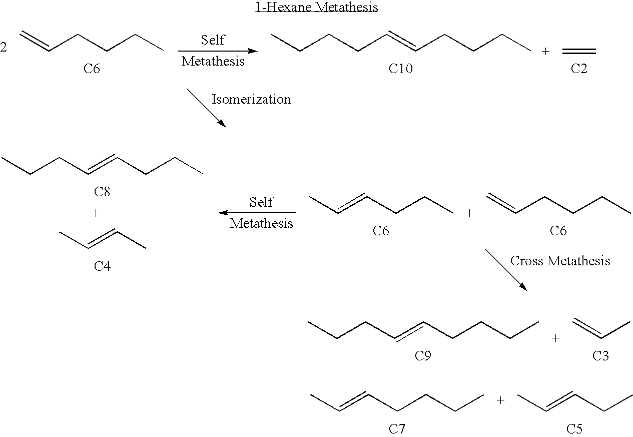

Supported rhenium catalysts may be used to catalyze olefin metathesis. However, since rhenium is a relatively expensive metal it is desirable to minimize the rhenium content of the catalyst while maintaining sufficient activity. Catalyst activity is usually compromised at low, such as less than 5 wt % rhenium content. This problem is commonly overcome through the addition of a suitable promoter, such as a tetraalkyltin compound. Xu Xiaoding et al discloses in J. Chem. Soc., Chem. Commun., 273-275(1986,) the use of mixed molybdenum oxide and rhenium oxide catalysts supported on alumina using a tetraalkyltin compound such as SnMe₄ as co-catalyst/promoter. While this approach of adding tin compounds may improve catalyst activity, the addition of environmentally unfriendly tin compounds may also be considered undesirable on an industrial scale.

Guo Xienxian et al discloses in J. Molecular Catalysis, 46 (1988) 119-130, a process for metathesis using a catalyst containing γ-alumina supported mixed rhenium and molybdenum oxides catalyst having a BET surface area of 185 m² g⁻¹. The process operates at a relatively high temperature of about 473° K (200° C.).

It is therefore desirable to obtain a metathesis catalyst having enhanced stability, high selectivity in olefin metathesis, low percentage of branching reaction due to condensation reaction or skeletal isomerization, low percentage of double bond isomerization and low gum formation due to polymerization of olefins, while having high activity at a relatively low operating temperature.

### SUMMARY OF THE PRESENT INVENTION

The invention provides a catalyst composition comprising: (a) rhenium, b) one or more metal(s) from Columns 5 and 6 of the Periodic Table, and (c) a support made from an alumina; wherein the surface area of the catalyst is at least 200 m²/g as determined by ASTM D-3663-03.

The invention also provides a metathesis process comprising contacting a feedstock comprising one or more olefins with the catalyst composition of this invention.

### BRIEF DESCRIPTION OF THE DRAWINGS

FIG. 1 is a graph which compares the percentage of conversion of 1-butene metathesis over time utilizing the mixed metal Catalysts B and C of the present invention with that of a comparative Catalyst A.

### DETAILED DESCRIPTION OF EMBODIMENTS

The present invention provides a catalyst having a relatively low rhenium content while having an enhanced activity and a high selectivity for an olefin metathesis reaction.

In one embodiment of the present invention, the catalyst composition comprises (a) rhenium, b) one or more metal(s) from Columns 5 and 6 of the Periodic Table, and (c) a support made from an alumina, preferably a γ-alumina. The support may be based on an alumina. In particular, the support (also known as carrier) may comprise (i) alumina and/or (ii) a composition made from a mixture comprising silica and alumina. While not intending to be bound by the theory, the composition made from a mixture comprising silica and alumina may be designated as silica/alumina or an aluminosilicate. The surface area of the catalyst is at least 200 m²/g as determined by ASTM D-3663-03. In a specific embodiment, the rhenium content is from about 0.5 to about 20 wt %, particularly from about 1.5 to about 12 wt %, more particularly from about 2.5 to about 6.0 wt %, and still more particularly from about 2.5 to about 4.0 wt % of rhenium metal based on the total weight of the catalyst. In a particular embodiment, the catalyst further comprises from about 0.5 to about 10 wt %, particularly from about 2 to about 7, more particularly from about 3 to about 5 wt % of one or more metal(s) from Columns 5 and 6 of the Periodic Table, including chromium, molybdenum, tungsten, vanadium, niobium and tantalum. As a specific embodiment, the Columns 5 and 6 metal contained in the catalyst is molybdenum.

As an embodiment of the present invention, the catalyst comprises from about 60.0 to about 98.6 wt %, particularly from about 70.0 to about 99.0, more particularly from about 73.5 to about 95.0, and still more particularly from about 84.5 to about 92.2 wt % of a support; particularly a support comprising an alumina or a support comprising (i) alumina and/or (ii) a composition made from a mixture comprising silica and alumina, more particularly a support comprising gamma alumina. Where a composition made from a mixture of silica and alumina is used, the support comprises from about 0.2 to about 10.0, particularly from about 1.0 to about 3.0, more particularly from about 1.5 to about 2.5 wt % silica.

In an embodiment of the present invention, the support has a surface area of at least 200, particularly at least 210, more particularly at least 220, and still more particularly at least 260 m²/g (square meters per gram), particularly not more than 500 or no more than 400 m²/g. As used herein, the surface area of the support or the catalyst is as determined by ASTM D-3663-03 based on calculation by the Brunauer-Emmett-Teller (BET) Method. The median pore diameter of the support is approximately from about 50 Å to about 150 Å, particularly from about 65 to about 100 Å, as determined by the mercury pore size distribution based on ASTM D-4222.

As used herein, the wt % of a metal of the catalyst refers to the percentage by weight of the metal (not the weight percentage of the metal compound) based on the total weight of the catalyst; and the wt % of the support refers to the percentage by weight of the alumina compound or a composition made from a mixture of silica and alumina compound based on the total weight of the catalyst. The total weight percentages of all ingredients of the catalyst add up to 100 weight percent.

As an embodiment of the present invention, the gamma alumina support employed for the present catalyst may be any suitable commercially available or any suitably prepared pseudo-boehmite material, and it may contain up to 10 wt % silica. Non-limiting examples of the suitable supports include Versal alumina from UOP, Baton Rouge, La., U.S.A., and Catapal aluminas from Sasol North America Inc., Houston, Tex., U.S.A. The support may be prepared by mulling (i) the above mentioned pseudo-boehmite material with (ii) a suitable amount of water, (iii) optionally a peptizing agent such as nitric acid, and (iv) optionally metal(s) and/or metal compound(s) from Columns 5 and 6 of the Periodic Table and/or rhenium-containing compound(s). In a particular embodiment, the support is prepared without metal(s) and/or metal compound(s) of Columns 5 and 6 and/or rhenium-containing compound(s) in the above mulled mixture and any Columns 5 and 6 metal(s) and/or rhenium-containing compound(s) contained in the catalyst is added after the support has already been prepared. In another particular embodiment, the support is prepared with at least a portion or all of the metal(s) and/or compound(s) of metal(s) from Columns 5 and 6 of the Periodic Table and/or rhenium metal and/or rhenium-containing compound(s) in the complete catalyst composition. Suitable Columns 5 and 6 metals include, but not limited to, any suitable organic or inorganic Columns 5 and 6 metal(s) and/or metal compound(s), particular metal oxides. One illustrative non-limiting example of the suitable Columns 5 and 6 compound(s) may be ammonium molybdates. The mulled mixture is then extruded to form extrudates of suitable sizes and shapes. The resulting extrudates are dried at a temperature in the range from about 250° C. to 350° C., followed by calcination, at a temperature from about 400° C. to 900° C., particularly from about 500 to about 700° C. As a particular non-limiting embodiment, the mulled support contains about 2 to about 10 wt % or about 4 to about 9 wt % of Columns 5 and 6 metal(s), such as molybdenum.

The catalyst may be prepared any suitable method known to one skilled in the art. Particularly it may involve any of the following methods:

(1) Co-mulling of at least a portion of the one or more Columns 5 and 6 metal(s) with the support followed by impregnating rhenium;

(2) Co-impregnating both the rhenium-containing compound and one or more Columns 5 and 6 metal(s) on to the support;

(3) Co-mulling at least a portion of the one or more Columns 5 and 6 metal(s) and at least a portion of the rhenium-containing compound with the support, and impregnate the remainder of the metals by impregnation;

(4) Co-mulling of at least a portion of rhenium-containing compound with said support followed by impregnating the one or more Columns 5 and 6 metal(s) and any remaining rhenium-containing compound;

(5) Impregnating the one or more Columns 5 and 6 metal(s) followed by impregnating rhenium-containing compound on to the support; and

(6) Impregnating the rhenium-containing compound followed by impregnating the one or more Column 5 and 6 metal(s) on to the support.

In one embodiment of the present invention, the surface area of the catalyst is at least 200, particularly at least 210, more particularly at least 220, still more particularly more than 230, yet still more particularly more than 250 or more than 260, and still more particularly not more than 400 m (square meters per gram). As used herein, the surface area of the catalyst is as determined by ASTM D-3663-03. The ASTM D-3663-03 method is based on calculations by the BET method. The pore volume of the catalyst is less than about 2.0, particularly less than about 1.0, more particularly less than 0.75, and still more particularly not less than 0.5 cm³/g (cubic centimeters per gram). As used herein, the pore volume of the catalyst is as determined by ASTM D-4222-03. The ASTM D-4222-03 method is based on the nitrogen desorption technique. The average pore diameter of the catalyst is from about 50 to about 150, particularly from about 60 to about 110 Å. As used herein, the average pore diameter of the catalyst is calculated from the pore volume (PV) and the surface area (SA) of the catalyst by dividing four times of the pore volume by the surface area, i.e. 4PV/SA.

The present catalyst containing mixed rhenium metal with Columns 5 and 6 metal(s) may be used to carry out a metathesis process at a relatively low temperature with minimal side reactions and hence high selectivity for products of metathesis reaction. In one particular embodiment of the present invention, the stability of the catalyst is improved over the catalyst having the same rhenium content but without Columns 5 and 6 metal(s). As used herein, the catalyst selectivity is defined as weight of the products from the metathesis reaction divided by total weight of the total products

The invention is further directed to a metathesis process which comprises providing a feedstock comprising one or more olefins and contacting the feedstock with a catalyst of the present invention as described above. The olefin feedstock employed herein preferably comprises one or more olefins having from two to 30 carbon atoms per molecule, and at least a portion of the charge has at least three carbon atoms per molecule. The feedstock may contain from four to 20 carbon atoms per molecule, or it may contain from four to 12 carbon atoms per molecule. The structure of the olefin may be a normal acyclic alpha-olefin, or an internal olefin or branched olefin. It may also be a cyclic olefin. The feedstock may contain at least one olefin selected from the group consisting of propylene, 1-butene, 2-butene, 1-pentene, 2-pentene, 2,4,4-trimethyl-2-pentene, 2,4,4-trimethy-1-pentene, 1-hexene, 2-hexene, 3-hexene, 2-heptene, 3-heptene, 1-octene, 2-nonene, 1-dodecene, 1-decene, 2-tetradecene, 1-hexadecene, 1-phenyl-2-butene, 4-octene, 3-eicosene, 2-methyl-4-octene, 4-vinylcyclohexene, 1,5,9,13,17-pentamethyloctadecene, and 8-cyclopentyl-4,5-dimethyl-1-decene. Illustrative and non-limiting examples include 1-butene metathesis to form ethylene and 3-hexene, 1-hexene metathesis to form ethylene and 5-octene, raffinate-2 metathesis, and cross metathesis of 2-butene with ethylene to produce propylene.

The process of the invention may be carried out either batch-wise or continuously, in liquid phase or gaseous phase, using a fixed catalyst bed, or a stirrer equipped reactor or other mobile catalyst contacting process as well as any other well known contacting technique. Preferred reaction conditions, e.g., temperature, pressure, flow rates, etc., vary somewhat depending upon the specific catalyst composition, the particular feed olefin, the desired products, etc.

The operable range of contact time for the process of this invention depends primarily upon the operating temperature and the activity of the catalyst, which is influenced by surface area, rhenium concentration and the Columns 5 and 6 metal concentration, activation temperature, etc.

In a particular embodiment, the present process is operated with a fixed-bed reactor in a continuous flow operation. The catalyst may be activated by first heating in air or an inert gas to a temperature from about 200° C. to about 1000° C., particularly from about 400° C. to about 600° C. for from about 0.5 hour to about 50 hours, particularly from about 2 to about 6 hours. The reactor is operated from about 0 to about 100° C., particularly from about 20 to about 50° C., more particularly from about 30 to about 40° C.; under a pressure of from about 0.05 MPa to about 4.05 MPa, particularly from about 0.09 MPa to about 0.6 MPa, more particularly from about 0.10 MPa to about 0.20 Mpa absolute, (normal atmospheric pressure is about 0.10 Mpa). Weight Hourly Space Velocity (WHSV) in the range of from about 0.5 to about 200 per hour, particularly from about 1 to about 40, more particularly from about 1 to about 10, and still more particularly from about 1 to about 3 per hour.

In one embodiment, from about 15 to about 70 wt %, particularly from about 40 to about 60% by wt of the olefin in the feedstock may be converted to metathesis products, when the feedstock is contacted with the catalyst for about 0.1 to about 4 hours. The selectivity of the process is from about 90 to about 100%, particularly from about 93 to about 99.5%, more particularly from about 95 to about 99%, when the feedstock is contacted with the catalyst for about 0.1 to about 4 hours. The molar ratio of RF/RP is from about 0.9 to about 1.0, particularly from about 0.95 to about 1.0, more particularly from about 0.99 to about 1.0,

Wherein,

RF is the molar ratio of branched olefins to normal olefins in the olefinic feedstock, and

RP is the molar ratio of branched olefins to normal olefins in the product stream.

In one particular non-limiting embodiment, the condensation reactions for a linear normal olefinic feed leading to branched species may be less than 4%, particularly less than 2% and still more particularly less than 1% on a molar basis based on the total moles of the products produced, branching due to skeletal isomerization may be less than 3%, particularly less than 2%, and more particularly less than 1% on a molar basis based on the total moles of the products produced. Double bond isomerization may be below 30%, particularly less than 20%, more particularly less than 10% on a molar basis based on the total products produced; and the gum from polyolefin formation may be less than 20 ppm, particularly less than 1 ppm based on the total weights of the products produced.

In one embodiment, the present process, using the present catalyst of rhenium in combination with metal(s) from Columns 5 and 6 of the Periodic Table, has the advantage of being operable at a low metathesis reaction temperature while maintaining high selectivity toward metathesis products, and having better stability and higher conversions/activities compared to rhenium-only catalysts with similar rhenium content. For this reason, it may suffice that the catalyst has a relatively low rhenium content. In a particular embodiment, the metathesis process is operable at from about 0 to about 100° C., particularly from about 20 to about 50° C., and more particularly from about 30 to about 40° C. The process also advantageously has improved low percentage of branching reaction due to condensation reaction or skeletal isomerization, low percentage of double bond isomerization and low polymer formation.

The invention will be illustrated by the following illustrative embodiments and comparative embodiments which are provided for illustration purpose only and are not intended to limit the scope of the instant invention.

### Illustrative Embodiment I—Preparation of Catlysts

I.A. Preparation of Catalyst A (˜3% Re on Alumina without Mo—Comparative)

2.16 grams of ammonium perrhenate (99+wt % purity, Aldrich Catalog Number 31,695-4) was dissolved in 50 ml of deionized water. This solution was added to 50 grams of a trilobe extrudate of high purity (purity close to 100%) gamma alumina, a surface area of approximately 260 m2/g, a median pore diameter approximately 97 Å (by Mercury Pore Size Distribution (PSD) ASTM D-4284-03), and less than 5% of the pore volume in pores with a diameter of greater than 350 Å. The alumina extrudate had been prepared from a pseudo-boehmite alumina powder produced by mixing an aqueous solution of aluminum sulfate (containing 27 wt % of aluminum sulfate (Al₂(SO₄)₃)) with an aqueous solution of sodium aluminate (containing 38.0 wt % sodium aluminate NaAlO₂) in a ratio to maintain the pH of mixture at about 8. The resulting alumina slurry was then washed and spray dried to yield an alumina powder containing approximately 88 wt % pseudo-boehmite (alumina monohydrate) and 12 wt % water. The powder was mulled with additional water added (totally about 60 wt % water based on the total weight of the entire mixture) and extruded. The extrudate was dried at about 150° C. and calcined at about 600° C. The water was subsequently removed from the catalyst by rotary evaporation. The catalyst was calcined for 4 hours at 500° C. under nitrogen to obtain Catalyst A. Catalyst A has a surface area of 243 m²/g as determined by ASTM D-3663-03, a pore volume is 0.66 cc/g measured by nitrogen adsorption based on ASTM D-4222-03 and an average pore diameter of 108.5 Å.

I.A1. Preparation of Catalyst A1 (˜7% Re on Alumina without Mo)

The catalyst was prepared following the same procedure as described in I.A. above, with the exception that 5.04 grams of ammonium perrhenate was used.

I.B. Preparation of Catalyst B (−3% Re/4% Mo Co-Mulled with Alumina)

A powder containing about 88 wt % pseudo-boehmite and about 12 wt % water was prepared according to U.S. Pat. No. 6,589,908, the entire description of which is herein incorporated by reference. The powder was prepared by mixing an aqueous solution of aluminum sulfate (containing 27 wt % of aluminum sulfate (Al₂(SO₄)₃)) with an aqueous solution of sodium aluminate (containing 38.0 wt % sodium aluminate NaAlO₂) in a ratio to maintain the pH of the mixture at about 9 in a two-step isothermal process first at 30° C. and then at about 60° C. The resulting alumina slurry was then washed and spray dried to yield an alumina powder containing approximately 88 wt % pseudo-boehmite (alumina monohydrate) and 12 wt % water. The powder was co-mulled, with Climax grade L MoO₃, with additional water added (totally about 60 wt % water based on the total weight of the entire mixture). The mixture was extruded, dried at about 150° C. and calcined at about 500° C. to give a molybdenum-containing support containing approximately 4% by weight molybdenum (which is approximately 6% by weight of molybdenum oxide). The extrudate was 1.3 mm trilobe and had a surface area of approximately 309 m²/g, a median pore diameter approximately 95 Å Mercury PSD, and less than 2% of the pore volume in pores with a diameter of greater than 350 Å. 2.16 grams of ammonium perrhenate (99+wt % purity, Aldrich Catalog Number 31,695-4) was dissolved in 50 ml of deionized water to form a solution. This solution was added to 50 grams of the above-described molybdenum-containing support. The water was removed by rotary evaporation. The catalyst was calcined for 4 hours at 500° C. under nitrogen. Catalyst B has a pore volume of 0.73 cc/gram, a surface area of 274 m²/g, and average pore diameter of 106.6 Å.

I.C. Preparation of Catalyst C (3% Re/4% Mo Impregnated on Alumina)

2.16 grams of ammonium perrhenate (99+wt % purity, Aldrich Catalog Number 31,695-4) and 4.08 grams of ammonium molybdate (99.98 wt %, Aldrich Catalog Number 27,790-8 were dissolved in 50 ml of deionized water. This solution was added to 50 grams of the high purity gamma alumina support as described in I.A. above. The water was removed by rotary evaporation. The catalyst was calcined for 4 hours at 500° C. under nitrogen. Catalyst C has a pore volume of 0.64 cc/gram, a surface area of 230 m²/g, and an average pore diameter of 108 Å.

I.C1. Preparation of Catalyst C1 (1% Re/4% Mo Impregnated on Alumina)

The catalyst was prepared following the same procedure as Described in I.C. above with the exception that 0.72 grams of ammonium perrhenate was used.

I.C2. Preparation of Catalyst C2 (6% Re/4% Mo Impregnated on Alumina)

The catalyst was prepared following the same procedure as Described in I.C. above with the exception that 4.32 grams of ammonium perrhenate was used.

I.D. Preparation of Support D (4 wt % Mo Co-Mulled with Alumina)

4000 grams of alumina containing 2% by weight of silica was co-mulled in a Simpson muller at ambient temperature for about an hour with 191.5 grams of molybdenum oxide purchase from Climax (L Grade), 5147 grams of de-ionized water and 90 grams of nitric acid. The mixture was then extruded, dried and calcined at 500° C. for 2 hours to convert the alumina from a mono-hydrate form to gamma alumina. The Support D contains about 4 wt % molybdenum (or approximately 6 wt % molybdenum oxide) and a surface area of approximately 320 m²/g and a median pore diameter of about 70 Å by mercury based on ASTM D4284-03.

I.D1. Preparation of Catalyst D (3% Re/4 wt % Mo Comulled with Alumina)

2.16 grams of ammonium perrhenate (99+wt % purity, purchased from Aldrich Catalog Number 31,695-4) was dissolved in 50 ml of deionized water. This solution was added to 50 grams of the Support D as described in I.D. above. The water was subsequently removed by rotary evaporation. The catalyst was calcined for 4 hours at 500° C. under nitrogen. Catalyst D has a pore volume of 0.64 cc/g, a surface area of 311 m²/g, and an average pore diameter of 82.4 Å.

I.D2. Preparation of Catalyst D1 (1% Re/4% Mo Co-Mulled with Alumina)

The catalyst was prepared using the same procedure as Described in I.D. above with the exception that 0.72 grams of ammonium perrhenate was used.

I.D3. Preparation of Catalyst D2 (6% Re/4% Mo Co-Mulled with Alumina)

The catalyst was prepared following the same procedure as Described in I.D. above with the exception that 4.32 grams of ammonium perrhenate was used. Catalyst D2 has a pore volume of 0.62 cc/g, a surface area of 294 m²/g and an average pore diameter of 68 Å.

### Illustrative embodiment II—Metathesis of 1-Butene

The catalysts A, B, and C were evaluated for the metathesis of 1-butene. Each catalyst (5.5 g) was loaded into a separate standard, tubular fixed-bed reactor. The catalyst was activated by first heating to 500° C. in flowing air for four hours then allowed to cool to room temperature under flowing nitrogen. The reactor was then heated to 35° C. The flow of gaseous 1-butene was then started at a WHSV of 1 and a pressure of 0.136 MPa (19.70 psi). Samples of the reactor effluent were taken periodically and analyzed by an on-line gas chromatograph. The catalyst selectivity is defined as weight of the desired products (ethylene+hexenes) divided by total weight of the total products (ethylene+propylene+pentenes+hexenes+heavier hydrocarbons). The conversion is defined as the reduction of the amount of 1-butene in the reactor product compared to the feed (feed is 100% 1-butene). The conversion (an indication of catalyst activity) and selectivity data for all three catalysts are given in Tables 1 and 2 below. Additionally, the product distribution for the run with Catalyst B is given in Table 3.

TABLE 1
1-Butene Metathesis - Conversion (%).

| Time | Catalyst A | | |
|---|---|---|---|
| (hr) | Comparative | Catalyst B | Catalyst C |
| 26 | 21.5 | 22.8 | 25.9 |
| 48 | 18.9 | 22 | 24.1 |
| 75 | 14.7 | 20.2 | 22.8 |
| 93 | 13.7 | 19.8 | 21.1 |

TABLE 2
Metathesis of 1-Butene - Selectivity (%).

| Time | Catalyst A | | |
|---|---|---|---|
| (hr) | Comparative | Catalyst B | Catalyst C |
| 26 | 97.3 | 97.4 | 96.8 |
| 48 | 97.1 | 97.6 | 96.8 |
| 75 | 96 | 97.6 | 96.9 |
| 93 | 96 | 97.5 | 96.8 |

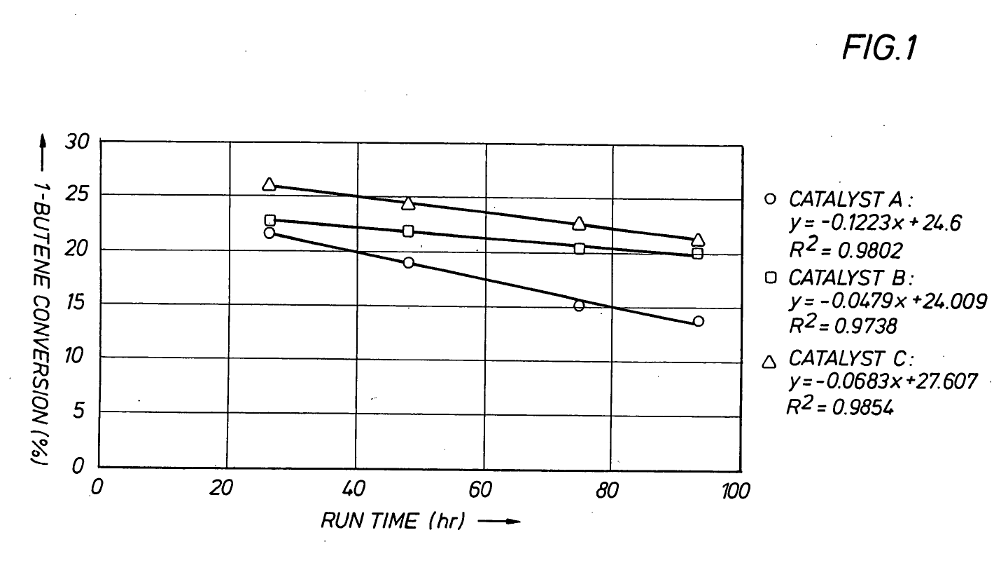

The data in Table 1 shows that all three catalysts are active for the metathesis of 1-butene. However, the decline of activity, as indicated by the declining conversion over time, is much greater for the rhenium-only catalyst (Comparative Catalyst A). In fact, 1-butene conversion declines linearly for all three catalysts over time, as illustrated in FIG. 1. The conversion over time for catalyst A plotted in FIG. 1 is represented by the equation y=−0.1223x+24.6 (R²=0.9802); that for Catalyst B is represented by the equation y=−0.0479x+24.099 (R²=0.9738); and that for Catalyst C is y=−0.0683+27.607 (R²=0.9854). As used herein, “y” is the percentage of 1-butene converted and “x” is the run time (hours). The value of R denotes how much the data points bear a linear relationship in the figure. For all three equations, the value R is very close to one, which means that the data points for each catalyst relate to each other close to a linear relationship. The slopes of the trend lines in FIG. 1 give a simple measure of these decline rates, showing that Catalyst A loses activity at approximately twice the rate of Catalysts B and C. Thus, the mixed-metal catalysts display much greater stability in 1-butene metathesis.

The data in Table 2 shows that all catalysts have the high selectivity characteristic of metathesis catalysts incorporating rhenium. This data also shows that the molybdenum present in Catalysts B and C is not contributing to double-bond isomerization of the 1-butene. Double-bond isomerization leads to the formation of byproduct propylene, pentene, and C7+olefins, which is undesirable for this application.

TABLE 3
Metathesis of 1-Butene
Product Distribution and Performance - Catalyst B (3% Re/4% Mo Co-Mulled with Alumina)

| Time Hr | Ethylene | Propylene | 1-butene | 2-butene | Pentenes | Hexenes | C7+ | % Conv | Selectivity |
|---|---|---|---|---|---|---|---|---|---|
| 3.8 | 2.7 | 2.7 | 66.7 | 0.1 | 3.3 | 24.1 | 0.0 | 33.2 | 81.6 |
| 8.3 | 3.3 | 0.4 | 71.5 | 0.0 | 0.4 | 24.1 | 0.0 | 28.5 | 97.4 |
| 12.8 | 3.1 | 0.3 | 72.2 | 0.0 | 0.3 | 23.7 | 0.0 | 27.8 | 97.6 |
| 17.3 | 3.5 | 0.3 | 73.9 | 0.0 | 0.3 | 21.7 | 0.0 | 26.1 | 97.6 |
| 21.7 | 3.4 | 0.3 | 75.1 | 0.0 | 0.3 | 20.5 | 0.0 | 24.9 | 97.6 |
| 26.2 | 3.1 | 0.3 | 77.2 | 0.0 | 0.3 | 18.7 | 0.0 | 22.8 | 97.4 |
| 30.6 | 2.6 | 0.3 | 74.5 | 0.0 | 0.3 | 22.0 | 0.0 | 25.5 | 97.8 |
| 35.0 | 2.6 | 0.3 | 76.6 | 0.0 | 0.2 | 19.9 | 0.0 | 23.4 | 97.7 |
| 39.5 | 2.2 | 0.3 | 76.9 | 0.0 | 0.2 | 20.0 | 0.0 | 23.1 | 97.7 |
| 44.0 | 2.5 | 0.3 | 77.6 | 0.0 | 0.2 | 19.1 | 0.0 | 22.4 | 97.7 |
| 48.5 | 2.5 | 0.3 | 78.1 | 0.0 | 0.2 | 18.6 | 0.0 | 21.9 | 97.6 |
| 52.9 | 2.3 | 0.3 | 78.0 | 0.0 | 0.2 | 18.8 | 0.0 | 22.0 | 97.7 |
| 57.4 | 2.4 | 0.3 | 78.2 | 0.0 | 0.2 | 18.5 | 0.0 | 21.8 | 97.7 |
| 61.9 | 1.8 | 0.3 | 79.3 | 0.0 | 0.2 | 18.1 | 0.0 | 20.7 | 97.5 |
| 66.4 | 1.9 | 0.3 | 79.1 | 0.0 | 0.2 | 18.1 | 0.0 | 20.9 | 97.5 |
| 70.8 | 1.7 | 0.3 | 79.4 | 0.0 | 0.2 | 18.1 | 0.0 | 20.6 | 97.6 |
| 75.3 | 1.9 | 0.3 | 79.8 | 0.0 | 0.2 | 17.5 | 0.0 | 20.2 | 97.6 |
| 79.7 | 1.9 | 0.3 | 79.7 | 0.0 | 0.2 | 17.6 | 0.0 | 20.3 | 97.6 |
| 84.1 | 1.7 | 0.3 | 80.0 | 0.0 | 0.2 | 17.5 | 0.0 | 20.0 | 97.6 |
| 88.6 | 1.7 | 0.3 | 80.4 | 0.0 | 0.2 | 17.1 | 0.0 | 19.6 | 97.5 |
| 93.1 | 1.6 | 0.3 | 80.2 | 0.0 | 0.2 | 17.4 | 0.0 | 19.8 | 97.4 |
| 97.5 | 1.9 | 0.3 | 81.2 | 0.0 | 0.2 | 16.1 | 0.0 | 18.8 | 97.3 |
| 102.0 | 1.7 | 0.3 | 82.6 | 0.0 | 0.2 | 14.8 | 0.0 | 17.4 | 96.9 |
| 106.5 | 2.3 | 0.3 | 84.7 | 0.0 | 0.1 | 12.2 | 0.0 | 15.3 | 96.7 |
| 111.0 | 2.2 | 0.3 | 84.4 | 0.0 | 0.2 | 12.6 | 0.0 | 15.6 | 96.8 |
| 115.5 | 2.0 | 0.3 | 84.8 | 0.0 | 0.2 | 12.3 | 0.0 | 15.2 | 96.7 |
| 120.0 | 1.9 | 0.3 | 85.2 | 0.0 | 0.2 | 12.1 | 0.0 | 14.8 | 96.6 |
| 124.5 | 1.8 | 0.3 | 86.1 | 0.0 | 0.2 | 11.3 | 0.0 | 13.9 | 96.4 |
| 128.9 | 1.7 | 0.3 | 85.7 | 0.0 | 0.2 | 11.7 | 0.0 | 14.3 | 96.5 |
| 133.4 | 1.6 | 0.3 | 86.1 | 0.0 | 0.2 | 11.5 | 0.0 | 13.9 | 96.4 |
| 137.9 | 1.5 | 0.3 | 85.9 | 0.0 | 0.2 | 11.8 | 0.0 | 14.1 | 96.5 |
| 142.4 | 1.5 | 0.3 | 86.0 | 0.0 | 0.2 | 11.7 | 0.0 | 13.9 | 96.4 |
| 146.9 | 1.4 | 0.3 | 86.4 | 0.0 | 0.2 | 11.4 | 0.0 | 13.6 | 96.4 |
| 151.3 | 1.3 | 0.3 | 86.9 | 0.0 | 0.2 | 11.0 | 0.0 | 13.1 | 96.3 |
| 155.7 | 1.2 | 0.3 | 87.6 | 0.0 | 0.2 | 10.4 | 0.0 | 12.4 | 96.1 |
| 160.2 | 1.2 | 0.3 | 87.7 | 0.0 | 0.2 | 10.3 | 0.0 | 12.3 | 96.1 |
| 164.7 | 1.1 | 0.3 | 87.8 | 0.0 | 0.2 | 10.2 | 0.0 | 12.1 | 96.1 |
| 169.1 | 1.0 | 0.3 | 87.9 | 0.0 | 0.2 | 10.3 | 0.0 | 12.0 | 96.1 |
| 173.6 | 1.0 | 0.3 | 88.4 | 0.0 | 0.2 | 9.8 | 0.0 | 11.6 | 96.0 |
| 178.1 | 0.9 | 0.3 | 88.4 | 0.0 | 0.2 | 9.9 | 0.0 | 11.6 | 96.1 |
| 182.6 | 0.9 | 0.3 | 88.5 | 0.0 | 0.2 | 9.9 | 0.0 | 11.5 | 96.1 |
| 187.1 | 0.8 | 0.3 | 88.4 | 0.0 | 0.2 | 10.0 | 0.0 | 11.5 | 96.1 |
| 191.6 | 0.7 | 0.3 | 88.6 | 0.0 | 0.2 | 9.8 | 0.0 | 11.3 | 96.2 |
| 196.0 | 0.7 | 0.3 | 89.2 | 0.0 | 0.2 | 9.3 | 0.0 | 10.7 | 96.0 |
| 200.5 | 0.7 | 0.3 | 89.6 | 0.0 | 0.2 | 9.0 | 0.0 | 10.4 | 96.0 |

### Illustrative Emboidment IIII—Metathesis of 1-Hexene

The catalysts A, A1, C, C1, C2, D, D1, and D2 were evaluated for the metathesis of 1-hexene. Each catalyst (1 g) was loaded into a separate standard, tubular, single pass, fixed-bed reactor. Each catalyst was activated by first heating to 500° C. in flowing air for four hours then allowed to cool to room temperature under flowing nitrogen. The reactor was then heated to 30-35° C. The flow of liquid 1-hexene was then started at a WHSV of 1 and a pressure of 1.38 MPa (200 psig). Samples of the reactor effluent were taken periodically and analyzed by an off-line gas chromatograph.

The catalyst selectivity was determined based on weight of the desired products (5-decene) divided by total weight of the liquid metathesis products (C7-C9, C11+).

The conversion is defined as the percentage of 1-hexene in feed minus the percentage of 1-hexene in the reactor product (feed is 100% 1-hexene).

The relative conversion and selectivity data, which are calculated by dividing the conversions and selectivities of various catalysts with that of Catalyst A (a 3% rhenium on alumina catalyst, without molybdenum) at the same conditions, are provided in Table 4 and Table 5. Thus, any catalyst showing a relative conversion and/or selectivity higher than 1 would be an improvement over Catalyst A.

The data in Table 4 shows that the mixed metal catalysts have higher activity at equivalent rhenium loadings. The improved stability is indicated by higher activity over time.

The data in Table 5 shows that the addition of molybdenum does not lead to reduced selectivity. That is, there is substantially no or very minimal, if any, increase in the double-bond isomerization activity of the catalysts due to the incorporation of molybdenum thus there should be no or very minimal production of undesired metathesis products from double-bond isomerization.

From Table 4, it is shown that several of the mixed metal catalysts have higher activity than the 3% Re standard catalyst. However, most of the catalysts show the same activity (e.g. Catalyst D and Catalyst D₂) even though the rhenium loadings are different. This is due to the fact that the metathesis of 1-hexene is an equilibrium-limited reaction. That is, thermodynamics limits the conversion of 1-hexene, and once the thermodynamic limit is reached, it is impossible to distinguish activity differences when the conversions are compared. In order to distinguish some of the more active catalysts, the reaction conditions were altered by increasing WHSV to 2. Under these conditions, twice as much 1-hexene must be converted to reach equilibrium. For these experiments, the standard catalyst was switched to 7% Re on alumina, which had comparable conversion to the best mixed metal catalysts in Table 4. The results from these tests are shown in Table 6. This data shows that the mixed metal catalysts can deliver better performance even at less than half of the rhenium loading of the standard catalyst. Again, the improved stability is indicated by the higher activity over time.

TABLE 4
Metathesis of 1-Hexene - Relative Conversion

| | | | Cat D2 | Cat D1 | Cat D3 | Cat C1 | Cat C | Cat C2 |
|---|---|---|---|---|---|---|---|---|
| | Cat A | Cat A1 | 1% Re/ | 3% Re/ | 6% Re/ | 1% Re/ | 3% Re/ | 6% Re/ |
| Time (hr) | 3% Re | 7% Re | 4% Mo | 4% Mo | 4% Mo | 4% Mo | 4% Mo | 4% Mo |
| 5 | 1.0 | 1.1 | 1.1 | 1.1 | 1.1 | 1.0 | 1.1 | 1.1 |
| 10 | 1.0 | 1.1 | 0.9 | 1.0 | 1.1 | 0.9 | 1.1 | 1.1 |
| 15 | 1.0 | 0.9 | 0.6 | 0.9 | 1.0 | 0.6 | 1.0 | 1.0 |
| 20 | 1.0 | 0.8 | 0.5 | 0.8 | 0.9 | 0.5 | 0.9 | 1.0 |
| 25 | 1.0 | 1.0 | 0.5 | 1.2 | 1.2 | 0.9 | 1.2 | 1.2 |
| 30 | 1.0 | 1.5 | 0.5 | 1.5 | 1.5 | 1.0 | 1.4 | 1.4 |
| 35 | 1.0 | 1.5 | 0.5 | 1.8 | 1.8 | 1.2 | 1.8 | 1.7 |
| 40 | 1.0 | 1.9 | 0.5 | 1.9 | 2.0 | 1.1 | 1.9 | 1.7 |

TABLE 5
Metathesis of 1-Hexene - Relative Selectivity

| | | | Cat D1 | Cat D2 | Cat D3 | Cat C1 | Cat C | Cat C2 |
|---|---|---|---|---|---|---|---|---|
| | Cat A | Cat A1 | 3% Re/ | 1% Re/ | 6% Re/ | 1% Re/ | 3% Re/ | 6% Re/ |
| Time (hr) | 3% Re | 7% Re | 4% Mo | 4% Mo | 4% Mo | 4% Mo | 4% Mo | 4% Mo |
| 5 | 1.0 | 1.0 | 1.0 | 1.0 | 1.0 | 1.0 | 1.0 | 1.0 |
| 10 | 1.0 | 1.0 | 1.0 | 1.0 | 1.0 | 1.0 | 1.0 | 1.0 |
| 15 | 1.0 | 1.0 | 1.0 | 1.0 | 1.0 | 1.0 | 1.0 | 1.0 |
| 20 | 1.0 | 1.0 | 0.9 | 1.0 | 1.0 | 1.0 | 1.0 | 1.0 |
| 25 | 1.0 | 1.0 | 1.0 | 1.0 | 1.0 | 1.0 | 1.0 | 1.0 |
| 30 | 1.0 | 1.0 | 1.0 | 1.0 | 1.0 | 1.0 | 1.0 | 1.0 |
| 35 | 1.0 | 1.1 | 0.9 | 1.0 | 1.0 | 1.0 | 1.1 | 1.1 |
| 40 | 1.0 | 1.1 | 1.0 | 1.0 | 1.0 | 1.1 | 1.0 | 1.1 |

TABLE 6
Relative Conversion at WHSV = 2

| Time | Cat A1 | Cat D1 | Cat D3 | Cat C2 |
|---|---|---|---|---|
| (hr) | 7% Re | 3% Re/4% Mo | 6% Re/4% Mo | 6% Re/4% Mo |
| 5 | 1.0 | 1.1 | 1.1 | 1.1 |
| 10 | 1.0 | 1.2 | 1.3 | 1.3 |
| 15 | 1.0 | 1.2 | 1.4 | 1.3 |
| 20 | 1.0 | 1.1 | 1.3 | 1.4 |
| 25 | 1.0 | 1.4 | 1.4 | 1.4 |
| 30 | 1.0 | 1.5 | 1.5 | 1.3 |
| 35 | 1.0 | 1.5 | 1.6 | 1.4 |
| 40 | 1.0 | 1.5 | 1.6 | 1.3 |

All elements and features described individually in the instant specification as well as all combinations thereof are contemplated as embodiments of the present invention. The ranges and limitations provided in the instant specification and claims are those which are believed to particularly point out and distinctly claim the instant invention. It is, however, understood that other ranges and limitations that perform substantially the same function in substantially the same manner to obtain the same or substantially the same result are intended to be within the scope of the instant invention as defined by the instant specification and claim.

We claim:

1. A catalyst composition comprising: (a) rhenium, (b) one or more metal(s) from Columns 5 and 6 of the Periodic Table, and (c) a support made from an alumina; wherein surface area of the catalyst is at least 200 m²/g as determined by ASTM D-3663-03.

2. The catalyst composition of claim 1 wherein the support in (c) is made from a composition comprising (i) an alumina and/or (ii) a composition made from a mixture of silica and alumina.

3. The catalyst composition of claim 1 wherein the catalyst comprises:
(a) from about 0.5 to about 20 wt % of rhenium,
(b) from about 0.5 to about 10 wt % of one or more metal(s) from Columns 5 and 6 of the Periodic Table, and
(c) from about 60.0 to about 9-8.6 wt % of the support, based on the total weight of the catalyst.

4. The catalyst composition of claim 3 wherein the catalyst comprises:
from about 1.5 to about 12 wt % of rhenium,
from about 2 to about 7 wt % of one or more metal(s) from Columns 5 and 6 of Periodic Table, and
from about 73.5 to about 95.0 wt % of the support, based on total weight of the catalyst; wherein the surface area of the catalyst is at least 210 m²/g as determined by ASTM D-3663-03.

5. The catalyst composition of claim 4 wherein the catalyst comprises:
from about 2.5 to about 6.0 wt % of rhenium,
from about 3 to about 5 wt % of one or more metal(s) from Columns 5 and 6, and
from about 84.5 to about 92.2 wt % of the support, based on total weight of the catalyst; wherein the surface area of the catalyst is at least 220 m²/g as determined by ASTM D-3663-03.

6. The catalyst composition of claim 1 wherein the catalyst is prepared by a method comprising step(s) selected from the group consisting of:
(1) co-mulling of at least a portion of said metal(s) in (b) with said support in (c) followed by impregnating rhenium in (a);
(2) co-impregnating both rhenium of (a) and the metal(s) of (b);
(3) co-mulling of at least a portion of said metal in (b) and at least a portion of the rhenium in (a) with the support in (c);
(4) co-mulling of at least a portion of rhenium in (a) with said support in (c) followed by impregnating the metal(s) in (b);
(5) impregnating the metal(s) in (b) followed by impregnating rhenium of (a) on to the support; and
(6) impregnating the rhenium of (a) followed by impregnating the metal(s) in (b) on to the support.

7. The catalyst composition of claim 1 wherein said metal in (b) comprises molybdenum and said alumina in (c) comprises a γ-alumina.

8. A metathesis process comprising:
(1) providing a feedstock comprising one or more olefins, and
(2) contacting the feedstock with a catalyst comprising: (a) rhenium, (b) one or more metal(s) from Columns 5 and 6 of the Periodic Table, and (c) a support made from an alumina;
wherein the surface area of the catalyst is at least 200 m²/g as determined by ASTM D-3663-03.

9. The metathesis process of claim 8 wherein the support in (c) is made from a composition comprising (i) an alumina and/or (ii) a composition made from a mixture of silica and alumina.

10. The metathesis process of claim 8 wherein, the catalyst comprises:
(a) from about 0.5 to about 20 wt % of rhenium,
(b) from about 0.5 to about 10 wt % of one or two metals from Columns 5 and 6 of the Periodic Table, and
(c) from about 60.0 to about 98.6 wt % of the support based on the total weight of the catalyst.

11. The metathesis process of claim 8 wherein the feedstock contains at least one olefin selected from the group consisting of propylene, 1-butene, 2-butene, 1-pentene, 2-pentene, 2,4,4-trimethyl-2-pentene, 2,4,4-trimethy-1-pentene, 1-hexene, 2-hexene, 3-hexene, 2-heptene, 3-heptene, 1-octene, 2-nonene, 1-dodecene, 1-decene, 2-tetradecene, 1-hexadecene, 1-phenyl-2-butene, 4-octene, 3-eicosene, 2-methyl-4-octene, 4-vinylcyclohexene, 1,5,9,13,17-pentamethyloctadecene, and 8-cyclopentyl-4,5-dimethyl-1-decene.

12. The metathesis process of claim 8 wherein said process is operated at from about 0 to about 100° C., from about 0.05 to about 4.05 MPa, and from about 0.5 to about 200 per hour Weight Hourly Space Velocity (WHSV).

13. The metathesis process of claim 8 wherein the feedstock is contacted with the catalyst for about 0.1 to about 4 hours.

14. The metathesis process of claim 8 wherein from about 15 to about 70 wt % of the olefin in the feedstock is converted to metathesis products, and the selectivity of the process is from about 90 to about 100% when the feedstock is contacted with the catalyst for about 0.1 to about 4 hours.

15. The metathesis process of claim 8 wherein the molar ratio of RF/RP is from about 0.9 to about 1.0, wherein,
RF is the molar ratio of branched olefins to normal olefins in the olefinic feedstock, and
RP is the molar ratio of branched olefins to normal olefins in the product stream.

16. The metathesis process of claim 8 wherein said metal in (b) comprises molybdenum and said support in (c) comprises a γ-alumina.

17. The metathesis process of claim 8 wherein said feedstock comprises 1-butene.

18. The metathesis process of claim 8 wherein said feedstock comprises 1-hexene.

19. The metathesis process of claim 8 wherein the branched species produced by the condensation reaction is less than 4% on a molar basis based on the total moles of the products produced, the branched species produced by skeletal isomerization is less than 3% on a molar basis based on the total moles of the products produced, and the double bond isomerization is below 30% on a molar basis based on the total products produced.

20. The metathesis process of claim 19 wherein the branched species produced by the condensation reaction is less than 2% on a molar basis based on the total moles of the products produced, the branched species produced by skeletal isomerization is less than 2% on a molar basis based on the total moles of the products produced, and the double bond isomerization is below 20% on a molar basis based on the total products produced.
